(19) United States
(12) Patent Application Publication
(10) Pub. No.: US 2024/0016650 A1
(43) Pub. Date: Jan. 18, 2024

(54) External Nasal Dilator for Delivering Scent to the Nose

(71) Applicant: ASO LLC, Sarasota, FL (US)

(72) Inventors: Brooke A. Hagerty, Bradenton, FL (US); Edmund A. Sinda, Bradenton, FL (US)

(21) Appl. No.: 18/221,928

(22) Filed: Jul. 14, 2023

Related U.S. Application Data
(60) Provisional application No. 63/389,462, filed on Jul. 15, 2022.

(51) Int. Cl. A61F 5/08; A61M 11/04; A61M 21/02; A61F 13/58; A61F 13/84

(52) U.S. Cl. CPC A61F 5/08; A61M 11/04; A61M 21/02; A61F 13/58; A61F 13/8405; A61M 2021/0016

(57) ABSTRACT

A nasal dilator includes a nose attachment member having an upper section that applies a recoil force to a nasal cavity of a human nose via adhesive attachment thereto. The nose attachment member also has a lower section including an arm that extends downwardly from the upper section to a nostril opening when the upper section is adhered to the wearer's nose. The arm has a scented portion.

### CROSS-REFERENCE TO RELATED APPLICATION

This claims the benefit of priority from Application No. 63/389,462, filed Jul. 15, 2023, which is incorporated by reference in its entirety.

### FIELD

This relates to the field of nasal dilators and, more particularly, to scented nasal dilators.

### BACKGROUND

External nasal dilators help with breathing through the nose. They gently expand the nasal passages by applying an outward force that prevents the nostrils from collapsing inwardly when the wearer inhales. Such dilators are composed of a resilient strip of material attached to an adhesive substrate. When the dilator is adhered to the skin of the nose the resilient material acts as a spring and applies an outward force to keep the left and right nostrils from collapsing during inhalation.

### BRIEF SUMMARY

Although external nasal dilators are very popular and effective, there is a need to improve them by providing additional functionality beyond merely applying mechanical forces to the nostrils.

An example of an apparatus designed to meet this need is a nasal dilator which includes a nose attachment member having an upper section. The upper section is configured to apply a recoil force to a nasal cavity of a human nose. The recoil force is provided via adhesive attachment of the nose attachment member to the human nose. The nasal dilator further includes a lower section, which includes an arm that extends downwardly from the upper section to a nostril opening when the upper section is adhered to the nose. The arm has a scented portion on it.

The nasal dilator may also include one or more of the following features.

The scented portion of the dilator may be positioned proximal to a terminal end of the arm.

A terminal end of the arm may extend over and/or into the nostril opening when the upper section is adhered to the nose.

The arm may be curved in such a way that an upper end of the arm, proximal to the upper section, curves outwardly away from a center of the upper section, and a lower end of the arm curves inwardly toward the center.

The scented portion of the dilator may include a scent member attached proximal to a terminal end of the arm. The scent member may be scented.

The scented portion of the dilator may include a scent member including a scented material. The scented material may be a gel, a liquid absorbent material, or both.

The arm of the dilator may include a shapeable material, which is capable of being formed into a shape, that is thereafter maintained by the material.

The arm of the dilator may extend down an ala of the nose when the upper section is adhered to the nose. The scented portion may also be bent upwardly toward the nostril opening.

An example of a method of delivering scent to a human nose includes adhering an external nasal dilator to a human nose with a left nasal passage and a right nasal passage. The dilator applies a recoil force to the left nasal passage and the right nasal passage. The dilator has a left arm and a right arm extending from it. The method also includes positioning the left arm proximal to a left nostril of the nose. This is done in such a way that a scented portion of the left arm is over and/or inside the left nostril. The method also includes positioning the right arm proximal to a right nostril of the nose. This is done in such a way that a scented portion of the right arm is over and/or inside the right nostril.

The method may also include one or more of the following features.

The method may include positioning the scented portions proximal to a terminal end of the left arm and right arm, respectively.

The left arm and right arm, respectively, may be curved in such a way that an upper end of the left arm or right arm proximal to the external nasal dilator curves outwardly away from a center of the dilator and a lower end of the arm curves inwardly toward the center.

The scented portions may be positioned proximal to a terminal end of the left arm and right arm, respectively. The scented portions may also include a scented material attached to the respective left arm or right arm.

The scented portions may include a scent member with a scented material. The scented material may be a gel, a liquid absorbent material, or both.

The left arm and right arm may extend down a left ala and a right ala, respectively, of the nose. The scented portions may also be bent upwardly toward the left nostril and right nostril, respectively.

Another example of a nasal dilator includes a nose attachment member that is attachable to a human nose. The nose attachment member includes a left end region that is adhereable to an outer wall of a left nasal passage and a right end region that is adhereable to an outer wall of a right nasal passage. An upper section of the nose attachment member includes a resilient member, which extends from the left end region to the right end region. The resilient member is configured to apply a recoil force to the left nasal passage and right nasal passage. A lower section of the nose attachment member includes a left arm, which extends downwardly from the upper section to a left nostril opening when the upper section is adhered to the nose. The left arm also has a left scented portion proximal to a terminal end of the left arm. The lower section also includes a right arm, which extends downwardly from the upper section to a right nostril opening when the upper section is adhered to the nose. The right arm has a right scented portion proximal to a terminal end of the right arm.

The nasal dilator may also include one or more of the following features.

A terminal end of the left arm and a terminal end of the right arm, respectively, of the dilator, may extend over and/or into the left nostril opening or right nostril opening when the left end region and right end region are adhered to the nose.

The left arm and right arm, respectively, may be curved in such a way that an upper end of the left arm or right arm proximal to the upper section curves outwardly away from a center of the upper section, and a lower end of the left arm or right arm curves inwardly toward the center.

The right scented portion and left scented portion, respectively, may include a scent member that carries a scented material. The scented material may be a gel, a liquid absorbent material, or both.

When the upper section of the dilator is adhered to the nose, the left arm may extend down a left ala of the nose, and the right arm may extend down a right ala of the nose. The left scented portion may be bent upwardly toward the left nostril opening, and the right scented portion may be bent upwardly toward the right nostril opening.

### BRIEF DESCRIPTION OF THE DRAWINGS

FIG. 1 is a diagram of an example of the nasal dilator being worn on a human nose.

FIG. 2 is a front view of the nasal dilator.

FIG. 3 is a back view of the nasal dilator.

FIG. 4 is an exploded side view of the nasal dilator.

FIG. 5 is a partial cutaway view of a human nose wearing an example of the nasal dilator.

FIG. 6 is a partial cutaway view of a human nose wearing an example of the nasal dilator.

FIG. 7 is a partial cutaway view of a human nose wearing an example of the nasal dilator.

FIG. 8 is a partial side view of an example of the nasal dilator showing a close up view of part of the lower end of the arm.

FIG. 9 is a partial side view of another example of the nasal dilator showing a close up view of part of the lower end of the arm.

FIG. 10 is a partial side view of another example of the nasal dilator showing a close up view of part of the lower end of the arm.

FIG. 11 is a front view of several examples of the nasal dilator showing additional possible arm configurations.

FIG. 12 is a front view of several examples of possible designs of the scent member.

### DETAILED DESCRIPTION

This disclosure describes certain examples, but not all possible examples of the nasal dilator and related methods. Where a particular feature is disclosed in the context of a particular example, that feature can also be used, to the extent possible, in combination with and/or in the context of other examples. The nasal dilator and related methods may be embodied in many different forms and should not be construed as limited to only the features and examples described here.

The external nasal dilators described here improve conventional external nasal dilator technology by delivering a scent to the nose while also mitigating collapse of the nasal wall tissue during inhalation.

The scent may provide aromatherapy or a medicated vapor. Accordingly, the scent may be an aroma that causes the wearer to experience a pleasurable state or the scent may have a therapeutic benefit such as efficacy in mitigating the symptoms of nasal congestion and helping the wearer breathe easier through the nose.

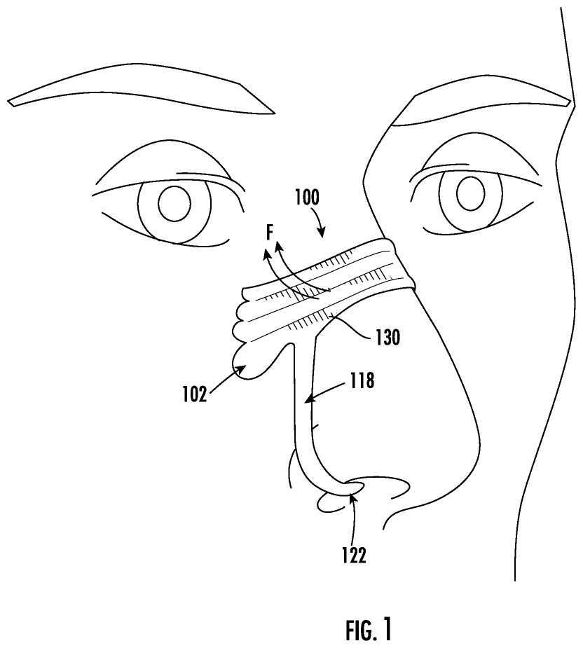

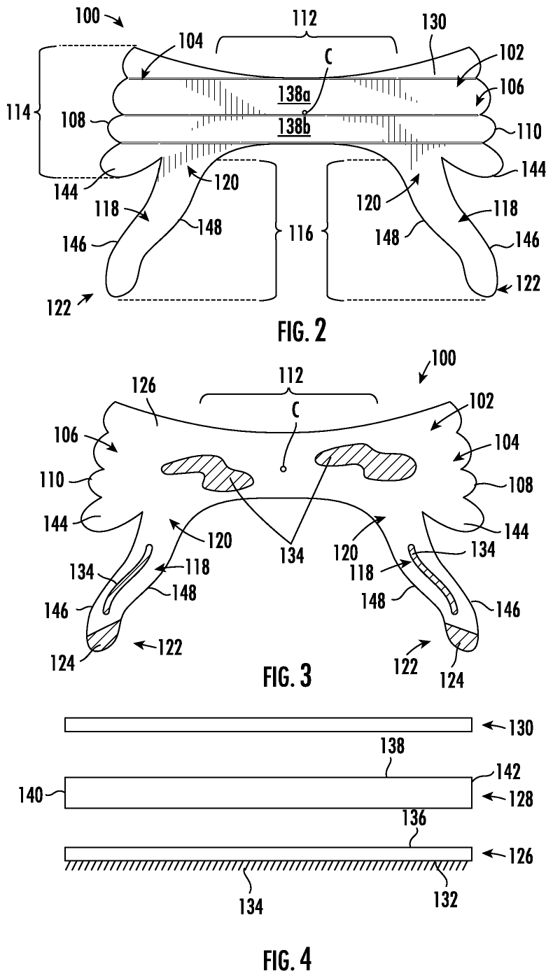

Referring to FIGS. 1-3, an example of the external nasal dilator 100 includes a nose attachment member 102 having an elongated shape and which exists in a substantially planar state when not being worn by a wearer. The nose attachment member 102 extends laterally between a first end region 104 and a second end region 106 that, respectively, define a left outer lateral edge 108 and right outer lateral edge 110 of the nose attachment member 102. The first end region 104 and second end region 106 are designed to affix the nose attachment member 102 to opposed sides of the nose over the wearer's nasal passages.

The nose attachment member 102 also includes an intermediate region 112 that extends laterally between the first end region 104 and second end region 106. The intermediate region 112 is designed to traverse the bridge B of the wearer's nose between the first end region 104 and second end region 106.

When adhered to the outer wall of the nose, the nasal dilator 100 applies a recoil force F to the outer wall, which lifts the outer wall tissue of the nostrils and dilates the internal nasal passages. Dilating the internal nasal passages reduces airflow resistance and increases airflow through the nasal passages when breathing.

The nasal dilator 100 includes an upper section 114 sized to fit across the wearer's nose and engage the outer skin of the nose and a lower section 116 extending downwardly from the upper section 114 along the wearer's nose to the nostrils. The lower section 116 includes a pair of arms 118 having an upper end 120 connected to the upper section 114 and a lower end 122 including a scented portion 124 that is positioned either directly under the nostrils or within the nostrils when the dilator 100 is worn.

Referring to FIGS. 1-4, the nose attachment member may be composed of a plurality of layers including a base layer 126, resilient layer 128, and cover layer 130.

The base layer 126 and cover layer 130 may be composed of thin, flexible material that is comfortable. Such a material may include, for example, woven or non-woven fabric, such as polyester, polyethylene, polypropylene, polyurethane, or the like. In the alternative, the material may include a plastic woven or non-woven material, such as polyethylene, polypropylene, polyurethane, or the like. In other examples, the material may be a foam, silicone, or the like.

The base layer 126 includes a nose engagement side 132 having adhesive 134 thereon for adhering the nose attachment member 102 to the nose. In certain examples, the adhesive 134 covers substantially the entire nose engagement side on the upper section 114 and the arms 118.

The adhesive 134 may be an adhesive suitable for skin contact. The adhesive material is selected from skin contact adhesives that can adhere firmly to the skin, but are removable without damaging the skin. Examples of skin contact adhesives include acrylic adhesives, silicone adhesives, hydrogels, hydrocolloids, silicone, or the like. The adhesive material may also include an additive that provides benefits to the skin such as vitamins, vitamin E, and/or zinc oxide, for example.

The base layer 126 includes a resilient layer engagement side 136 adapted to engage with the resilient layer 128 such that the resilient layer 128 cooperatively mates with the base layer 126. The resilient layer engagement side 136 may include adhesive or the like that is capable of attaching the resilient layer 128 to the base layer 126.

In some particular examples, the base layer 126 has a thickness of about 0.5 to about 500 μm. The base layer 126 may be oxygen and/or moisture permeable or impermeable, depending on the materials chosen.

The resilient layer 128 includes at least one resilient member 138 that extends laterally between the first end region 104 and second end region 106. The resilient member 138 may extend completely to the left and right lateral edges 108, 110 or may terminate in the first end region 104 and second end region 106 short of the left and right lateral edges 108, 110.

The resilient layer 128 may include a single resilient member 138, or multiple resilient members 138. In the example shown, a first resilient member 138a and a second resilient member 138b extend substantially parallel in the lateral direction of the nose attachment member 102. The resilient members 138 have a first end 140 and a second end 142.

Resilient members 138 may be made from any suitable material having the desired flexibility and resiliency to generate the desired amount of recoil force, such as metal, plastic, or the like. The recoil force is the spring biasing force created by the resiliency of the resilient members. The resilient members are typically flat, semi-rigid, and resilient so as to generate a recoil force when flexed across the nose, which is what lifts the outer wall tissues of the nostrils. This recoil force is typically between about 15 and 35 grams, or about 25 grams. In a particular example, the resilient members 138 are made of biaxially oriented polyester with a thickness of 0.1 mm to 0.3 mm and a width of 0.3 mm to 0.6 mm. The thickness of the resilient members 138 may be selected to obtain a nasal dilator with the desired amount of recoil force. Other examples of materials from which a resilient member 138 can be made include polyethylene, polypropylene, or the like.

The resilient members 138 may be secured to the resilient layer engaging side 136 of the base layer 126 by adhesive material thereon. The adhesive material may cover the resilient layer engaging side 136 or may be substantially the same size and shape as the resilient members 138.

The number and design of the resilient member(s) 138 may depend on the desired amount of force to be applied to the nose, the direction of force to be applied to the nose, and aesthetic considerations.

The resilient member(s) 138 may be disposed between the base layer 126 and cover layer 130 and held in place by an adhesive, which also stops the base layer 126 and cover layer 130 from separating when the dilator 100 is being worn or when the dilator 100 is being removed.

The cover layer 130 is primarily made of a thin, flexible material. The cover layer 130 may be made of the same material as the base layer 126 or a different material from the base layer 126. The cover layer 130 and base layer 126 may be laminated together using adhesive and/or a thermally fusable film. The cover layer 130 may have a thickness of about 1 to about 500 μm and may be oxygen and moisture permeable or impermeable.

When the nose attachment member 102 is adhered to the wearer's nose, the recoil force from the resilient members 138 draws the outer wall tissue of the left and right nasal passages outward. The flexibility of the base layer 126, resilient layer 128, and cover layer 130 allows the nose attachment member 102 to substantially conform to the unique curves of the nose of each individual wearer.

The arms 118 are attached to the upper section 114 at an upper end 120 thereof by being integrally formed with the upper section 114. In alternate examples, the arms 118 may be non-integral with the upper section 114.

The arms 118 are positioned proximal to right and left end regions 104, 106 of the upper section 114. Between each arm 118 and the respective outer lateral edges 108, 110 is a tab 144 that extends laterally beyond an outer edge 146 of the arm 118. The outer edge 146 and an inner edge 148 of the arm 118 are curved to provide the arm 118 a curvature that permits the arm 118 to lay flat on the curvature of the nose. The arm 118 is curved such that the upper end 120 curves outwardly away from the center C of the upper section 114 and the lower end 110 curves inwardly toward the center C. The scented portion 124 at the lower end 122 is further curved inwardly so that the scented portion 124 can extend laterally across the base of the nose over and/or into the nostril opening.

The scented portion 124 may be positioned at different locations on the lower end 122. Referring to FIGS. 5-7, a few of the many examples of how the scented portion 124 may be positioned are provided by showing their location in a cross section of a human nose with the septum S, ala A, and nasal passages P indicated for reference. When the dilator 100 is worn, each arm 118 is adhered to the outer skin of the nose extending downwardly from the upper section 114 toward the base of the nose.

In the example of FIG. 5, at the base of the nose, the scented portion 124 is bent upwardly in such a way that the scent member 150 attached to the scented portion 124 extends upward into the nostrils beyond the septum S.

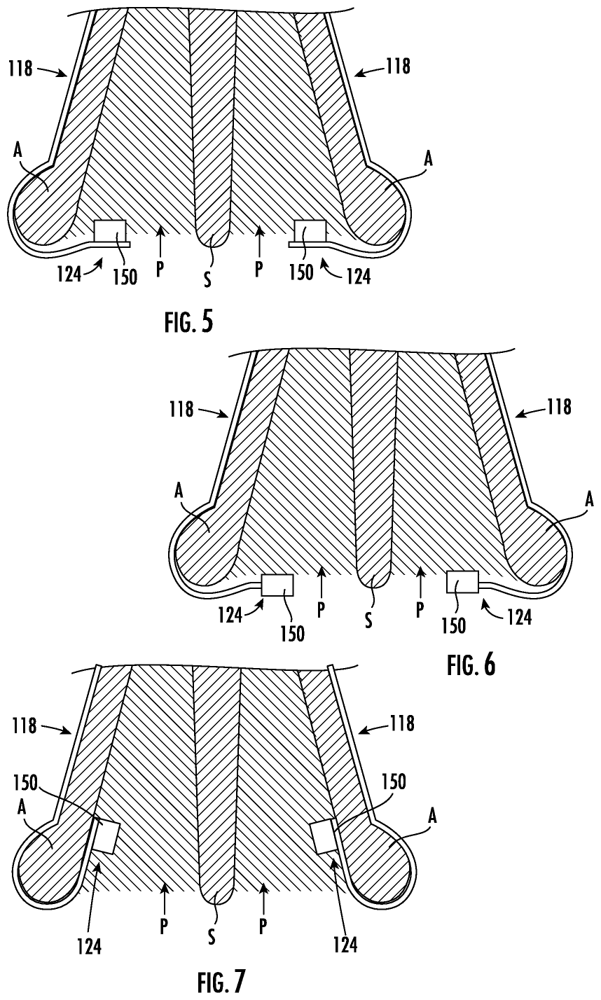

In the example of FIG. 6, at the base of the nose, the scented portion 124 is bent upwardly in such a way that the scent member 150 attached to the scented portion 124 extends laterally across the nose toward the septum S. The scent member 150 is positioned directly beneath the respective nostril.

In the example of FIG. 7, at the base of the nose, the scented portion 124 is bent upwardly in such a way that the scent member 150 attached to the scented portion 124 extends upward into the nostrils beyond the septum S and contacts the interior wall of the respective nostril. In this case, adhesive 134 on the base layer 126 may adhere the scented portion 124 to the inside of the nostril.

The scent member 150 may have many different forms, depending on the desired application. In one example, the scent member 150 is a portion of a scent formula that is applied manually to the scent member 150 by the wearer using an applicator. In this case, the wearer can select a desired scent and apply a scent formula to the scent member 150. The scent formula may be obtained from a scent formula container. An amount of the scent formula may be applied to the scent member 150 using an applicator such as a cotton swab, a finger, a dropper, a brush, or the like.

In another example, the scent member 150 is a liquid absorbent material having the scent formula distributed therein. The liquid absorbent material is capable of absorbing the scent formula and holding the scent formula therein. Examples of liquid absorbent materials include polyester, lyocell, wool, cotton, nylon, hydrocolloid, hydrogel, bamboo, microfiber, lycra, polyurethane, foam, or the like. The liquid absorbent material may also be capable of maintaining the scent formula therein when mucus contacts the scent member 150 or when the wearer blows or wipes the nose.

In yet another example, the scent member 150 is a gel with the scent formula distributed therein. The gel is capable of containing the scent formula and holding the scent formula therein. Examples of gel materials include hydrocolloids, hydrogels, or the like.

The scent formula is a chemical composition that includes a scent compound. The scent compound may be therapeutically effective for mitigating nasal congestion and/or coughing or may provide an aromatherapy benefit to the wearer. Examples of the scent formula may include the scent compound in a suitable liquid, liquid-like, wax, or wax-like carrier. Such carriers may include water, oil, oil-based, wax, or wax-based materials in which the scent compound is dissolved and may have a low viscosity similar to water or a high viscosity similar to a gel or waxy substance. Certain examples of the scent formula include essential oils and scented petrolatum products. Certain examples of the scent formula may include menthol, peppermint oil, eucalyptus, camphor, rosemary, lavender, cyprus, pine, cinnamon, any aromatherapy scent, or the like.

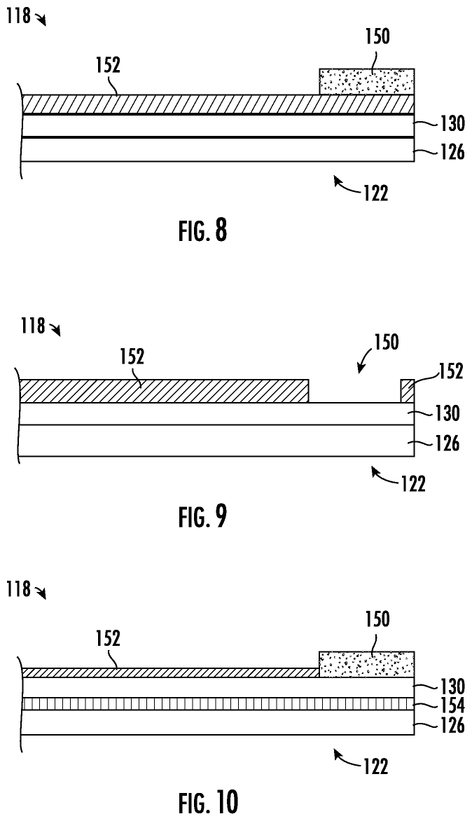

Additional details about the construction of the scented portion 124 will now be described by referring to FIGS. 8-10.

Referring to FIG. 8, the scented portion 124 may include the cover layer 130 with an adhesive layer 152 adhering the scent member 150 to the cover layer 130.

Referring to FIG. 9, the scent member 150 may be a portion of the cover layer 130 that is devoid of a section of the adhesive layer 152 relative to FIG. 8.

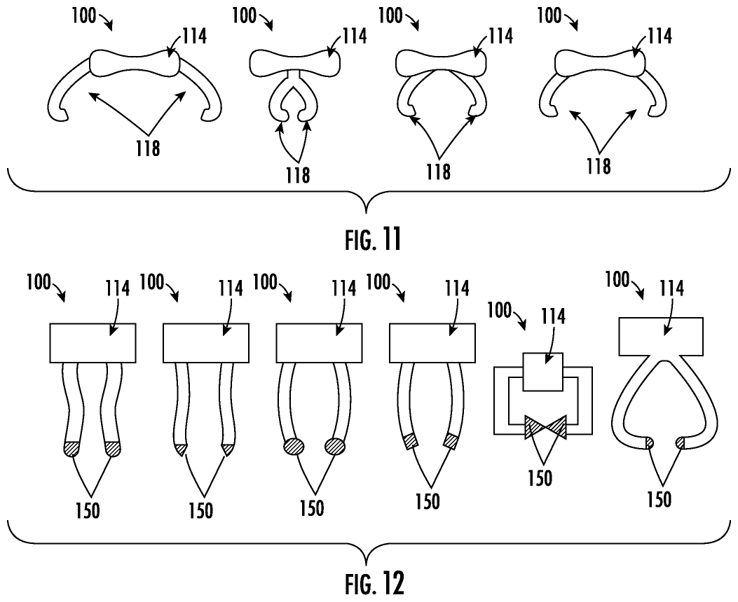

Referring to FIG. 10, certain examples of the arm 118 may be formable to a desired shape. In this example, the arm 118 includes a shapeable material layer 154 between the base layer 126 and cover layer 130. The shapeable material layer 154 includes a thin strip of shapeable material such as metal, plastic, foam, or the like. The shapeable material is able to be bent into position and maintain that position for a period of time without substantially deforming. This permits the position of the scent member 150 to be adjusted by the wearer.

The design of the arms 118 and upper section 114 in the drawings discussed above are provided by way of example only for the purpose of describing the details of a particular example. The designs have many other possible forms. FIG. 11 shows four additional examples of the dilator 100 with different arm 118 and upper section 114 configurations.

The design of the scent member 150 may also vary. FIG. 12 is a front view of several examples of the scent member 150 designs.

The dilator 100 may be used in a method of delivering scent to a human nose. The method includes applying the dilator 100 to the wearer's nose by pressing the adhesive 120 against the skin. The dilator 100 is positioned so that the scented portion 112 extends across or into the nostrils to deliver scent thereto when the wearer inhales.

The nasal dilator and related methods are not limited to the details and features described in connection with the example embodiments. There are numerous variations and modifications of the nasal dilator and methods that may be made without departing from the scope of what is claimed.

That which is claimed is:

1. A nasal dilator comprising a nose attachment member having (a) an upper section configured to apply a recoil force to a nasal cavity of a human nose via adhesive attachment of the nose attachment member to the human nose and (b) a lower section including an arm extending downwardly from the upper section to a nostril opening when the upper section is adhered to the nose, the arm having a scented portion thereon.

2. The nasal dilator of claim 1, wherein the scented portion is positioned proximal to a terminal end of the arm.

3. The nasal dilator of claim 1, wherein a terminal end of the arm extends over and/or into the nostril opening when the upper section is adhered to the nose.

4. The nasal dilator of claim 1, wherein the arm is curved in such a way that an upper end of the arm proximal to the upper section curves outwardly away from a center of the upper section and a lower end of the arm curves inwardly toward the center.

5. The nasal dilator of claim 1, wherein the scented portion includes a scent member attached proximal to a terminal end of the arm, the scent member being scented.

6. The nasal dilator of claim 1, wherein the scented portion includes a scent member including a scented material, the scented material being a gel and/or liquid absorbent material.

7. The nasal dilator of claim 1, wherein the arm includes a shapeable material capable of being formed into a shape and thereafter maintaining the shape.

8. The nasal dilator of claim 1, wherein, when the upper section is adhered to the nose, the arm extends down an ala of the nose and the scented portion is bent upwardly toward the nostril opening.

9. A method of delivering scent to a human nose, the method comprising:
adhering an external nasal dilator to a human nose having a left nasal passage and a right nasal passage, the external nasal dilator applying a recoil force to the left nasal passage and right nasal passage, the external nasal dilator having a left arm and a right arm extending therefrom;
positioning the left arm proximal to a left nostril of the nose in such a way that a scented portion of the left arm is over and/or inside the left nostril; and
positioning the right arm proximal to a right nostril of the nose in such a way that a scented portion of the right arm is over and/or inside the right nostril.

10. The method of claim 9, wherein the scented portions are positioned proximal to a terminal end of the left arm and right arm, respectively.

11. The method of claim 9, wherein the left arm and right arm, respectively, are curved in such a way that an upper end of the left arm or right arm proximal to the external nasal dilator curves outwardly away from a center of the external nasal dilator and a lower end of the arm curves inwardly toward the center.

12. The method of claim 9, wherein the scented portions are positioned proximal to a terminal end of the left arm and right arm, respectively, the scented portions including a scented material attached to the respective left arm or right arm.

13. The method of claim 9, wherein the scented portion includes a scent member including a scented material, the scented material being a gel and/or liquid absorbent material.

14. The method of claim 9, wherein the left arm and right arm respectively include a shapeable material capable of being formed into a shape and thereafter maintaining the shape.

15. The method of claim 9, wherein the left arm and right arm extend down a left ala and a right ala, respectively, of the nose and the scented portion is bent upwardly toward the left nostril and right nostril, respectively.

16. A nasal dilator comprising:
a nose attachment member that is attachable to a human nose, the nose attachment member including a left end region that is adhereable to an outer wall of a left nasal passage and a right end region that is adhereable to an outer wall of a right nasal passage;
an upper section of the nose attachment member including a resilient member extending from the left end region to the right end region, the resilient member being configured to apply a recoil force to the left nasal passage and right nasal passage; and
a lower section of the nose attachment member including (a) a left arm extending downwardly from the upper section to a left nostril opening when the upper section is adhered to the nose, the left arm having a left scented portion proximal to a terminal end of the left arm and (b) a right arm extending downwardly from the upper section to a right nostril opening when the upper section is adhered to the nose, the right arm having a right scented portion proximal to a terminal end of the right arm.

17. The nasal dilator of claim 16, wherein the terminal end of the left arm and the terminal end of the right arm, respectively, extend over and/or into the left nostril opening or right nostril opening when the left end region and right end region are adhered to the nose.

18. The nasal dilator of claim 16, wherein the left arm and right arm, respectively, is curved in such a way that an upper end of the left arm or right arm proximal to the upper section curves outwardly away from a center of the upper section and a lower end of the left arm or right arm curves inwardly toward the center.

19. The nasal dilator of claim 16, wherein the right scented portion and left scented portion, respectively, include a scent member including a scented material, the scented material being a gel and/or liquid absorbent material.

20. The nasal dilator of claim 16, wherein, when the upper section is adhered to the nose, the left arm extends down a left ala of the nose, the right arm extends down a right ala of the nose, the left scented portion is bent upwardly toward the left nostril opening, and the right scented portion is bent upwardly toward the right nostril opening.
